(19) United States
(12) Patent Application Publication
(10) Pub. No.: US 2006/0271062 A1
(43) Pub. Date: Nov. 30, 2006

(54) Method for reducing the risk of posterior capsular opacification

(76) Inventors: Paul L. Kaufman, Madison, WI (US); Baohe Tian, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/413,470

(22) Filed: Apr. 28, 2006

Related U.S. Application Data
(60) Provisional application No. 60/675,839, filed on Apr. 28, 2005.

(51) Int. Cl. A61F 9/013

(52) U.S. Cl. 606/107; 623/6.56; 514/912

(57) ABSTRACT

It is disclosed here that actin-disrupting compounds and 1-(5-isoquinolinyl-sulfonyl)-2-methylpiperazine can be used to facilitate the removal of lens epithelial cells from the lens capsule during cataract surgery and they may also be used to inhibit lens epithelial cells' proliferation and/or migration after surgery to reduce the risk or severity of posterior capsular opacification. Various related methods, kits, and ocular implants for reducing the risk or severity of posterior capsular opacification are provided.

### CROSS-REFERENCE TO RELATED APPLICATIONS

This application claims priority to U.S. provisional application Ser. No. 60/675,839, filed on Apr. 28, 2005, which is herein incorporated by reference.

### STATEMENT REGARDING FEDERALLY SPONSORED RESEARCH OR DEVELOPMENT

This invention was made with United States government support awarded by the following agencies: NIH EY02698. The United States has certain rights in this invention.

### BACKGROUND OF THE INVENTION

The most frequent complication of extracapsular cataract extraction and intraocular lens (IOL) implantation is posterior capsular opacification (PCO) caused by the proliferation and migration of residual lens epithelial cells toward the center of the posterior lens capsule, which obstructs the visual axis and causes a visual disturbance. A detailed description on structural relationship between lens epithelial cells and lens capsules as well as how PCO develops are provided in U.S. Pat. No. 6,186,148, which is herein incorporated by reference in its entirety. PCO is a type of secondary cataract that significantly affects the postoperative vision (Apple D J, et al. Surv Ophthalmol. 37:73-116, 1992).

PCO is commonly treated by neodymium-YAG laser capsulotomy, which provides a clear visual axis for the eye having PCO. Surgical capsulotomy has also been frequently performed in pediatric cases for the same purpose (Araiz J J, et al. Invest Ophthalmol Vis Sci. 34:522-530, 1993; Gimbel H V. J Cataract Refract Surg. 23:652-656, 1997). Although improved IOL design and surgical technique have reduced the incidence of PCO (Spalton D J. Eye. 13 (Pt 3b):489-492, 1999; Nishi O, et al. Ophthalmic Surg Lasers. 29:587-594, 1998; Olson R J, et al. J Cataract Refract Surg. 29:55-58, 1998), laser/surgical capsulotomy is still sometimes necessary and involves the risk of complications such as retinal detachment. In addition, the laser/surgical posterior capsulotomy may not be feasible for the next generation of IOLs (accommodating IOLs) that need an intact posterior capsule to function. Furthermore, despite reduced proliferation of lens epithelial cells by improved IOL design and surgical technique, even the best IOLs would still potentially induce anterior to posterior capsular adhesions to each other and to the IOL haptics. These changes could substantially limit the movement of the postoperative capsule and accommodating IOLs, and in turn could prevent the IOLs from functioning (McDonald J P, et al. Invest Ophthalmol Vis Sci. ARVO electronic abstracts, 2003. http://abstracts.iovs.org/). Another complication of extracapsular cataract extraction and IOL implantation is the growth of fibrous strands which could also induce capsular adhesions (McDonald J P, et al. Invest Ophthalmol Vis Sci. ARVO electronic abstracts, 2003. http://abstracts.iovs.org/).

Investigators are now searching for pharmaceutical means to inhibit the proliferation of lens epithelial cells and the growth of fibrous stands, which may not only reduce the possibility of PCO and the consequent destruction of the posterior capsule by laser/surgical capsulotomy, but also retain the capsule movement postoperatively. To date, mitomycin C, daunomycin, 5-fluorouracil, colchicine and daunorubicin have been found effective in inhibiting lens epithelial cell proliferation in vitro (Haus C M, et al. Br J Ophthalmol. 80:1087-1091, 1996; Ismail M M, et al. Ophthalmic Res. 28:64-69, 1996; Legler U F C, et al. J Cataract Refract Surg. 19:462-470, 1993; Pearson P A, et al. Invest Ophthalmol Vis Sci. 32:1235-1237, 1991; Ruiz J M, et al. Ophthalmic Res. 22:201-208, 1990). However, none of these pharmaceutical agents is routinely used clinically because of cytotoxic side effects on the corneal endothelium.

Kim, J T et al. (Exp. Eye Res. 74:585-594, 2002), incorporated herein by reference in its entirety, disclosed a study in which a disintegrin called salmosin was shown to inhibit lens epithelial cell proliferation and migration in rabbit eyes. U.S. Pat. No. 6,186,148, incorporated herein by reference in its entirety, disclosed the use of focal contact modulating compounds such as certain oxidants, urokinase receptor modulators, and proenzymes to help remove lens epithelial cells during cataract surgery for the purpose of preventing PCO.

There is still a need in the art to provide more agents that can reduce the risk of PCO by facilitating the removal of lens epithelial cells from the lens capsule during cataract surgery and/or to inhibit lens epithelial cell proliferation and migration after cataract surgery.

### BRIEF SUMMARY OF THE INVENTION

The present invention is summarized in that actin-disrupting compounds and 1-(5-isoquinolinyl-sulfonyl)-2-methylpiperazine (H-7) can be used to facilitate the removal of lens epithelial cells from the lens capsule during cataract surgery and they may also be used to inhibit lens epithelial cells' proliferation and/or migration after surgery to reduce the risk or severity of PCO. Various methods, kits, and ocular implants for reducing the risk or severity of PCO are provided herein.

### BRIEF DESCRIPTION OF THE DRAWINGS

FIG. 1 depicts the chemical structures of some of the actin-disrupting compounds and H-7.

### DETAILED DESCRIPTION OF THE INVENTION

The present invention is based on the inventors' discovery that actin-disrupting compounds, which predominantly loosen cell-cell adhesions versus cell-extracellular matrix adhesions, can be used to reduce the risk or severity of PCO in a human or non-human animal. Examples of such actin-disrupting compounds include but are not limited to latrunculins (e.g., latrunculin-A and latrunculin-B), swinholide-A, jasplakinolide, and cytochalasins (cytochalasin B and cytochalasin D) (Coue M, et al. FEBS Lett. 213:316-318, 1987; Lyubimova A, et al. J. Cell Biol. 65:469-478, 1997; Spector I, et al. Science. 219:493-495, 1983; Spector I, et al. Cell Motil Cytoskeleton. 13:127-144, 1989; Epstein D L, et al. Invest Ophthalmol. Vis. Sci. 40:74-81, 1999; Peterson J A, et al. Invest Ophthalmol Vis Sci. 40:931-941, 1999; Cai S, et al. Mol Vision. 6:132-143, 2000; Peterson J A, et al. Exp Eye Res. 70:307-313, 2000; Tian B, et al. Invest Ophthalmol Vis Sci. 42:3187-3192, 2001; Kaufman P L, et al. Invest. Ophthalmol. Vis. Sci. 23:646-650, 1982). It is further disclosed that the risk or severity of PCO in a human or non-human animal can also be reduced by H-7 (Birrell G B, et al. J Cell Physiol. 141:74-84, 1989; Bershadsky A, et al. Curr Biol. 6:1279-1289, 1996; Yu J C, et al. Microvasc Res. 43:100-111, 1992; Volberg T, et al. Cell Motil Cytoskel. 29:321-338, 1994; Tian B, et al. Arch Ophthalmol. 116:633-643, 1998; Gills J P, et al. Invest Ophthalmol Vis Sci. 39:653-658, 1998; Liu X, et al. Mol Vision. 7:145-153, 2001) when used in a manner similar to an actin-disrupting compound. For the purpose of the present invention, when a compound is referred to by its name, both the compound and pharmaceutically acceptable salts thereof are intended to be covered.

Without intending to be limited by theory, the inventors believe actin-disrupting compounds loosen lens epithelial cells from neighboring cells and thus make the residue lens epithelial cells easier to remove from the lens capsule by, for example, aspiration or other suitable methods during eye surgery such as extracapsular cataract extraction. Lens epithelial cells possess a network of polygonal arrays of actin microfilaments, to which cell-cell junctions interact through a complex submembrane interlinking “plaque.” Compounds which interfere with the formation of the actin microfilaments, promote disruption of the filaments, interfere with actin assembly into bundles, or disturb the relationship between actin and the plaque will disrupt cell-cell junctions and cause cells to pull apart from each other. For example, latrunculins A and B can alter cell shape and disrupt microfilament organization by sequestering G-actin, leading to disassembly of actin filaments and separations of cell-cell adhesions (Coué M, et al. FEBS Lett. 213:316-318, 1987; Lyubimova A, et al. J. Cell Biol. 65:469-478, 1997; Spector I, et al. Science. 219:493-495, 1983; Spector I, et al. Cell Motil Cytoskeleton. 13:127-144, 1989; Epstein D L, et al. Invest Ophthalmol. Vis. Sci. 40:74-81, 1999; Peterson J A, et al. Invest Ophthalmol Vis Sci. 40:931-941, 1999; Cai S, et al. Mol Vision. 6:132-143, 2000).

In addition, actin filaments in non-muscle cells such as lens epithelial cells are highly dynamic structures that are constantly assembled, disassembled, and reorganized as the cell changes its shape, divides, crawls, and adheres to neighboring cells or a substratum. Therefore, in addition to affecting cellular adhesions, inhibition of the actin filaments by actin-disrupting compounds are also expected to inhibit the division and migration of residual lens epithelial cells by inhibiting cellular contractibility and inducing microfilament depolymerization or deterioration. This also leads to reduced PCO risk or severity. In this regard, the actin disrupting compounds can coat an ocular implant such as an IOL or be provided in the lens capsule in a sustained release format to prevent PCO. Furthermore, stress fiber (bundles of actin filaments) formation and cytoskeleton-related contractility are main factors for the capsule wrinkling and tensing (Moisseiev J, et al. J Cataract Refract Surg. 15:531-533, 1989). Actin-inhibiting compounds may also improve postoperative vision by reducing lens capsule wrinkling and tensing through inhibiting the actin cytoskeleton in residual lens epithelial cells.

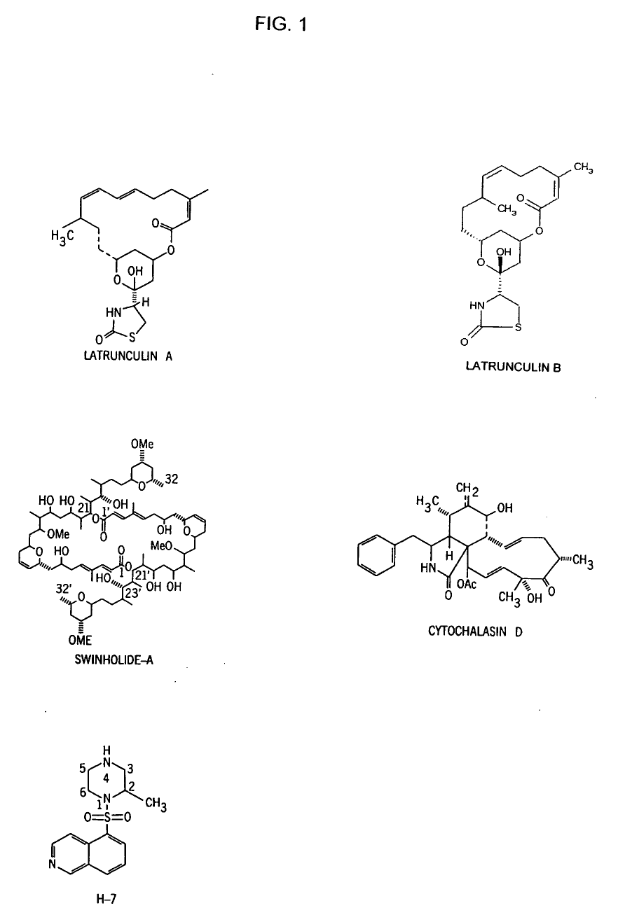

H-7, which is commercially available from Sigma, is a broad-spectrum serine-threonine-specific protein kinase inhibitor. By inhibiting myosin light chain kinase and Rho kinase, H-7 inhibits activation of the myosin light chain and thereby inhibits actomyosin-driven contractility. Accordingly, the present invention contemplates the use of H-7 to inhibit the division and migration of residual lens epithelial cells for reducing the risk or severity of PCO. A secondary effect of H-7 resulting from the inhibition of myosin light chain kinase and Rho kinase is the deterioration of the actin microfilament system and perturbation of its membrane anchorage and loss of stress fibers and focal contacts. In this regard, the present invention contemplates the use of H-7 to facilitate the removal of residue lens epithelial cells from the lens capsule by, for example, aspiration or other suitable methods during eye surgery such as extracapsular cataract extraction. H-7's structure is shown in FIG. 1. While H-7 and actin-disrupting compounds are expected to reduce the risk or severity of PCO by different mechanisms, one embodiment of the present invention contemplates using more than one actin-disrupting compounds and/or H-7 together.

It should be noted that actin-disrupting compounds as referred to in the present application selectively break cell-cell adhesions without completely or substantially breaking adhesions to the underlying extracellular matrix. Examples of such actin-disrupting compounds include but are not limited to latrunculins (e.g., latrunculin-A and latrunculin-B), swinholide-A, jasplakinolide, and cytochalasins (cytochalasin B and cytochalasin D).

Latrunculins are macrolides isolated from the marine sponge Latrunculia magnifica and they are specific and potent actin-disrupting agents that bind to monomeric G-actin to block its polymerization, leading to the disassembly of actin filaments (Couf et al., FEBS Lett. 213:316-318, 1987; Lyubimova et al., J. Cell Biol. 65:469-478, 1997; and Spector et al., Science 219:493-495, 1983). The two most common latrunculins, latrunculin-A and latrunculin-B, cause reversible dose- and incubation time-dependent destruction of actin bundles in several types of cultured cells including human trabecular meshwork cells (Couf et al., supra; Lyubimova et al., supra; Spector et al., Science 219:493-495, 1983; Spector et al., Cell Motil. Cytoskeleton 13:127-144, 1989; Epstein et al., Invest. Ophthalmol. Vis. Sci. 40:74-81, 1999; Peterson et al., Invest. Opthamol. Vis. Sci. 40:931-941, 1999; and Cai et al., Invest. Ophthamol. Vis. Sci. 40:S505. Abstract No. 2668, 1999).

Latrunculin-A is a 2-thiazolidinone macrolide isolated from the marine sponge Latrunculia magnifica. The sponges are prominent in and may be collected from the Red Sea, where they can be found at a depth of 6.0 to 30.0 meters. The sponges may be squeezed manually to collect the toxic fluid. A combination of Sephadex LH-20 and Silica-gel chromatographies allows one to get rid of the glycerides in the fluid and obtain three pure toxins, one of which is latrunculin-A which may be isolated via separation techniques known in the art. Latrunculin-A is also commercially available from Molecular Probes. The structure of latrunculin-A, an oil, is presented in FIG. 1 (Kashman et al., Tetrahedron Letters 21:3629-3632, 1980; and Spector et al., Cell Motil. Cytoskel. 13:127-44, 1989). Latrunculin-A binds monomeric G-actin in a 1:1 molar ratio, thereby shifting the equilibrium between G- and F-actin and preventing the nucleation and elongation of actin filaments, leading to destabilization of the actin filaments. See e.g., Spector et al., Cell Motil. & Cytoskel., 13:127-44, 1989.

Latrunculin-B has a similar structure (FIG. 1) and actin-disrupting activity in cultured cells to latrunculin-A (Coue et al., FEBS Lett. 213:316-318, 1987; Lyubimova et al., J. Cell. Biol. 65:469-478, 1997; and Spector et al., Science 219:493-495, 1983). Latrunculin-B is commercially available from Calbiochem.

Swinholide-A is a macrolide isolated from the marine sponge Theonella swinhoei; the sponge may be collected, among other places, from the Red Sea. Swinholide-A may by obtained from frozen Theonella swinhoei lyophilized and successively extracted with petrol-ether and CH₂Cl₂/MeOH (8:2). The latter extract may then be chromatographed on Sephadex LH-20(MeOH/CHCl₃ (1:1)) and on silica gel (petrol-ether, ether, ethyl acetate). The ethyl acetate fractions provide swinholide-A as a microcrystalline compound (melting point, 102° C., C₃₉H₆₆O₁₀.H₂O) (Carmely and Kashman, Tetrahedron Lett. 26:511-514, 1985; and Bubb et al, J. Biol. Chem. 270:3463-3466, 1995).

Swinholide-A has been described as a 44-membered dilactone macrolide with a 2-fold axis of symmetry. Its chemical structure is set forth in FIG. 1. Swinholide-A reportedly disrupts the actin cytoskeleton of cells grown in culture, sequesters actin dimers in vitro in both polymerizing and non-polymerizing buffers, and rapidly severs F-actin in vitro with high cooperativity (Bubb et al., J. Biol. Chem. 270:3463-3466, 1995). U.S. Pat. No. 6,586,425 disclosed that swinholide-A affects cells' actin filament and cell-cell adhesion.

Jasplakinolide, a macrolide derived from the Indo-Pacific marine sponge Jaspis johnstoni, is a cyclic peptide with a 15-carbon macrocyclic ring that contains three amino acid residues (Crews et al., Tetrahed. Lett. 27:2797-2800, 1986; and Zabriskie et al., J. Amer. Chem. Soc. 108:3123-3124, 1986). Jasplakinolide is a potent inducer of actin polymerization in rabbit skeletal muscle actin (Bubb et al., J. Biol. Chem. 269:14869-14871, 1994) and is believed to stabilize actin filaments by binding F-actin (e.g., similarly to phalloidin, a peptide isolated from the mushroom Amanita phalloides). Studies conducted with jasplakinolide suggest that inhibition of depolymerization by jasplakinolide can perturb the actin cytoskeleton similar to inhibition of polymerization as proper assembly of the actin cytoskeleton strongly depends on actin dynamics (Bubb et al, J Biol. Chem. 275:5163-5170, 2000; Senderowicz et al, J Natl Cancer Inst. 87:46-51, 1995; Spector et al, Microsc Res Tech. 47:18-37, 1999; Cramer, Curr Biol. 9:1095-1105, 1999; and Rotsch and Radmacher, Biophys J. 78:520-535, 2000).

The cytochalasins (e.g., cytochalasin B and D) are fungal metabolites that inhibit actin filament elongation (e.g., preventing G-actin polymerization by capping the filaments and preventing their growth) and promote actin filament nucleation, resulting in a decrease in average filament length, although the fraction of F-actin may remain constant or even increase. This decrease in length (but not net actin depolymerization) is likely responsible for the effects of cytochalasins on the actin cytoskeleton structure and cell morphology.

Various systems such as in vitro cell/organ culture systems and/or in vivo systems can be used to confirm that a particular compound is useful in practicing the present invention and a skilled artisan is familiar with these systems. Some of the systems are described in U.S. Pat. Nos. 6,586,425 and 6,186,148, Spalton D J. Eye 13 (Pt 3b):489-492, 1999 (the rabbit eye model), and Liu C S et al. Invest Ophthalmol & Vis Sci. 37:906-914, 1996 (the human lens capsular bag culture model), all of which are herein incorporated by reference in their entirety. For example, a skilled artisan is familiar with how to determine whether a compound is an actin-disrupting compound that can selectively inhibit cell-cell adhesion versus cell-extracellular matrix adhesion using in vitro cultured cells. In this regard, bovine aortic endothelial cells as well as a variety of other cells form, in culture, a continuous monolayer with well-developed intercellular junctions, extensive adhesion to the underlying matrix, and a well-developed network of actin microfilaments. The integrity of the monolayer can be determined by light- and electron-microscopy and the organization of the actin cytoskeleton by fluorescent labeling with fluorophore-conjugated phalloidin. The cells can be plated at half confluency on clean glass coverslips and cultured in complete medium for 2-4 days until they reached confluency. A compound can then be added to the culture medium for a predetermined periods of time, after which the cells can be fixed with a fixative appropriate for the specific antigen being examined. Fixed cells can then be either fluorescently labeled for actin using a fluorescein- or rhodamine-conjugated phalloidin or immunofluorescently labeled for other cytoskeletal or junctional proteins. Immunofluorescence microscopy and transmitted light microscopy can be conducted and the integrity of the microfilament system in control- and compound-treated cells can then be compared visually. At the resolution of the light microscope used (Axiophot, Zeiss Oberkochen, Germany), intact junctions typically appear as a single actin-, vinculin-, catenin-, and cadherin-rich “line.” Disruption of the junctions lead to disappearance of this line or its apparent division or “splitting” into two lines due to the dissociation of the junction. A digital microscopic system (Kam et al., J. Cell Sci. 108:1051-1062, 1995) can be used for quantitative analyses, morphometry, and 3-dimensional reconstruction.

Some in vivo models for determining whether a compound is useful in practicing the present invention are described in the examples below.

Preferred compounds for the purpose of the present invention include (a) macrolides such as latrunculins (e.g., latrunculin-A and latrunculin-B), swinholide-A, and jasplakinolide, (b) cytochalasins such as cytochalasin B and cytochalasin D, and (c) H-7. In some preferred embodiments, one or more of non-comeotoxic latrunculins such as latrunculin-A, latrunculin-B, and swinholide-A or the non-comeotoxic H-7 is used.

In one aspect, the present invention relates to a method for reducing the risk or severity of PCO in a human or non-human animal. The method involves providing access to lens epithelial cells attached to an interior surface of a lens capsule of an eye in the human or non-human animal and contacting the lens epithelial cells with an actin-disrupting compound or H-7 in an amount sufficient to reduce PCO. The compounds disclosed herein may be provided in a treatment composition that contains one or more of the compounds and at least one pharmaceutically acceptable carrier. When used during cataract surgery, the compounds help remove more lens epithelial cells from the lens capsule. When used after cataract surgery, the compounds inhibit proliferation and/or migration of the residual lens epithelial cells. The compounds may be used at either one or both stages.

When used at the surgery stage, the treatment composition is preferably provided in the form of a solution (treatment solution). A skilled artisan is familiar with the carriers or solutions that can be used. Examples of treatment solutions include but are not limited to those based on water, saline, phosphate-buffered saline, dextrose, glycerol, or the like, and combinations thereof. In addition, if desired the compositions may contain minor amounts of auxiliary substances such as wetting or emulsifying agents, stabilizing or pH buffering agents or preservatives. The treatment with the actin-disrupting compound or H-7 allows more lens epithelial cells to be removed from the lens capsule by a chosen cell removal process (e.g., a standard mechanical irrigation and/or aspiration process) than without such treatment. Preferably, the treatment allows at least 5%, 10%, 20%, 30%, 40%, 50%, 60%, 70%, 80%, 90%, or 95% more cells to be removed than without the treatment.

The appropriate amount of compound(s) in a treatment solution depends on the specific compound(s) and the human or non-human animals being treated. Examples of non-human animals that can be treated include but are not limited to primates, rodents, canines and felines. A skilled artisan can readily determine the optimal dosage of a particular agent for a particular treatment subject with routine experimentation. Non-limiting examples of dosage of various compounds include but are not limited to about 0.1 to about 10 micromolar of latrunculin-A, about 0.06 to about 5 micromolar of latrunculin-B, about 0.1 to about 5.0 micromolar of jasplakinolide, about 0.05 to about 1.0 micromolar of swinholide A, about 10 to about 200 micromolar of cytochalasin B, about 1 to about 20 micromolar cytochalasin D, and about 50 to about 500 micromolar of H-7. The term “about” is used to cover concentrations that slightly deviate from the recited concentration but retain the essential function of the recited concentration.

In one embodiment, the agents disclosed herein are used in connection with cataract surgery. Such use involves the steps of making an incision in the anterior part of an eye (e.g., the cornea, the sclera, or the corneal scleral junction), optionally introducing a viscoelastic material into the anterior chamber, making an opening in the anterior capsule of the lens (e.g., in the central area of the anterior capsule of the lens by performing an approximately 4-5 mm continuous curvilinear capsulorhexis), removing lens cortex and nucleus from the lens capsular bag or the eye (e.g., by phacoemulsification, laser energy, or simple delivery through the opening of the lens anterior capsule and the incision of the anterior part of the eye), contacting lens epithelial cells on the lens capsule with one or more compounds disclosed herein (e.g., inserting a fine needle or cannula into the lens capsular bag through the eye incision and the viscoelastic in the anterior chamber to conduct a bolus injection with a treatment solution and/or including the same compound(s) in the irrigation solution during the following irrigation and aspiration procedure), removing residual lens cortex fibers and lens epithelial cells from the lens capsule (e.g., by aspiration and irrigation with physiological solutions with or without compound(s)), inserting an ocular implant (e.g., an artificial IOL or an ocular ring) inside the lens capsular bag if applicable, and closing the incision in the anterior part of the eye. It is understood that some of these steps can be combined, the order of some of the steps can be changed, and additional steps may be included. For example, the compounds may be introduced into the lens capsule to contact lens epithelial cells before or after an opening on the lens capsule is made, before or after the lens nucleus is removed, and before or after the lens cortex fibers are removed. A viscoelastic material may optionally be introduced into the lens capsular bag before the ocular implant is inserted and washed out before the incision on the anterior part of eye is closed. A skilled artisan is familiar with all the details related to cataract surgery. Some of these details, such as how to introduce treatment solutions into the lens capsule, the appropriate volumes of the treatment solutions, various types of IOLs that can be used, and how to conduct aspiration for removing lens epithelial cells, are described in U.S. Pat. No. 6,186,148. When an opening or incision is made on a lens capsule, the lens capsule is often referred to as a “capsular bag.” For the purpose of the present invention, the terms “lens capsule” and “capsular bag” are used interchangeably to describe the capsular bag.

When a treatment solution is introduced into the lens capsule as described above, one or more calcium chelating agents such as ethylenediamine tetraacetic acid (EDTA) and ethylene glycol bis amino ethyl tetraacetic acid (EGTA) can be optionally included in the solution to secure passage of the treatment solution between the lens epithelial cells. A skilled artisan is familiar with the details of using the calcium chelating agents in this regard. See e.g., U.S. Pat. No. 6,186,148.

Many of the compounds described here such as latrunculin-B and H-7 are effective to facilitate the removal of lens epithelial cells at a concentration and time of exposure that is essentially not corneotoxic. Even if an agent is somewhat toxic, washing out the agent from the eye following a treatment should be sufficient to reduce the toxicity, if any, to an acceptable level. Other measures may also be taken. For example, prior to making an opening in the lens capsule or administering a treatment solution into the lens capsule, a viscoelastic material may be introduced into the anterior chamber to maintain the anterior chamber depth. The viscoelastic material may also prevent any of the substance escaping from the lens capsular bag and prevent damage to the corneal endothelium. If an inhibitor is known for any of the compounds disclosed herein, it may also be included in a washing solution or the viscoelastic material (see e.g., U.S. Pat. No. 6,186,148).

A dye can also be included in a treatment solution of the present invention so that the introduction of the solution to its intended site can be monitored. A skilled artisan is familiar with the dyes that can be used. See e.g., U.S. Pat. No. 6,186,148.

An actin-disrupting compound or H-7 can also be provided on the outer surface of an ocular implant such as an IOL or a capsular ring to reduce the risk or severity of PCO by inhibiting the proliferation and/or migration of the residue lens epithelial cells after cataract surgery. By “capsular ring,” we mean an artificial device to be inserted into the lens capsular bag, which includes, but is not limited to, capsular tension ring, capsule-bending ring, capsular edge ring (Morcher GmbH, Germany), endocapsular tension ring, equator ring, polymer implant (U.S. Pat. No. 5,618,553), device for stretching the crystalline capsule, capsular bag implants (U.S. Pat. No. 5,593,436) or the like. An ocular implant coated with an actin-disrupting compound or H-7 is within the scope of the present invention.

Otherwise, actin-disrupting compounds or H-7 can be provided in a sustained- or controlled-delivery formulation for inhibiting the proliferation and/or migration of residue lens epithelial cells after cataract surgery and various such formulations are known in the art. See e.g., Remington's Pharmaceutical Sciences, 17th Ed. 1985; Joshi, J. Ocul. Pharmacol. 10:29-45, 1994; McCalden et al., Experientia 46:713-15, 1990; Feist et al., J. Cataract Refract. Surg. 21:191-95, 1995; Cheng et al., Invest. Opthalmol. Vis. Sci. 36:442-53, 1995; and Chetoni et al., J. Ocul. Pharmacol. Ther. 129:245-52, 1996; all of the foregoing references are herein incorporated by reference. Techniques for formulating a variety of other sustained- or controlled-delivery means, such as liposome carriers, bio-erodible microparticles or porous beads and depot injections, are also known to those skilled in the art. See, for example, PCT/US93/00829 that describes controlled release of porous polymeric microparticles for the delivery of pharmaceutical compositions. Additional examples of sustained-release preparations include semipermeable polymer matrices in the form of shaped articles, e.g., films or microcapsules. Sustained release matrices may include polyesters, hydrogels, polylactides (U.S. Pat. No. 3,773,919 and EP 58,481), copolymers of L-glutamic acid and gamma ethyl-L-glutamate (Sidman et al., Biopolymers, 22:547-556, 1983), poly(2-hydroxyethyl-methacrylate) (Langer et al., J. Biomed. Mater. Res., 15:167-277, 1981 and Langer et al., Chem. Tech., 12:98-105, 1982), ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., Proc. Natl. Acad. Sci. (USA), 82:3688-3692, 1985; EP 36,676; EP 88,046; and EP 143,949. An ocular implant may be coated with an actin-disrupting compound or H-7 in a sustained- or controlled-delivery form.

In another aspect, the present invention relates to a kit for use in cataract surgery. The kit includes an artificial IOL (the artificial lens can be a lens with a preformed, fixed shape or a liquid polymer that will assume a shape once introduced into the lens capsule) and one or more compounds disclosed herein. Optionally, the kit can contain one or more of the following: a viscoelastic material, a calcium chelating agent (e.g., EDTA and EGTA), and a dye.

The invention will be more fully understood upon consideration of the following non-limiting examples.

### EXAMPLE 1

### Effect of Latrunculin-B on Residue Lens Epithelial Cells During Cataract Surgery

This example demonstrates that intracapsularly administering latrunculin-B during extracapsular lens extraction facilitates the clearance of residual lens epithelial cells and in turn prevents PCO.

Materials and Methods

Two rabbits were involved in this study (one for latrunculin-B experiment and one for vehicle experiment). For each rabbit, both eyes received extracapsular lens extraction and were treated with the same 2 μM latrunculin-B or vehicle (0.25% DMSO) during surgery. Following the extracapsular lens extraction, enucleation was performed. The rabbit was then sacrificed with an overdose of pentobarbital. The lens capsular bag was removed from the enucleated eye and then fixed with 4% paraformaldehyde. Micrographs were taken by a digital camera attached on a microscope from the fixed capsular bag.

The detailed procedures are as follows:

1. Slit lamp examination (SLE) was performed by a trained ophthalmologist 1 week before surgery to exclude rabbits that had ocular inflammation or other abnormalities.

2. Intramuscular injection (i.m.) with carprofen (1-5 mg/kg) was conducted once daily for one week.

3. Animal was anesthetized as follows: A pre-medication of 20-50 mg/kg i.m. ketamine, 2-5 mg/kg i.m. xylazine, and 0.01-0.05 mg/kg buprenorphine SQ (subcutaneous inoculation) were given; animals were masked with isoflurane to relax for intubation and then maintained at 1-5% isoflurane in O₂ during surgery.

4. Extracapsular lens extraction:

(1) Eye preparations: The pupil of the surgical eye was dilated with 1% cyclopentolate (Cyclogyl™), 0.3% flurbiprofen (Ocufen [symbol] ), 2.5% phenylephrine and 1% tropicamide (Mydriacyl™) (4 times at five-minute intervals); eyelashes were trimmed; the ocular area was disinfected with povidone iodine; and a wire lid speculum was inserted to retract the lids.

(2) Clear cornea approach: A corneal incision was made superiorly with a 3.0-3.2 mm keratome.

(3) Anterior capsulotomy: Viscoelastic (Provisc™) was injected to maintain anterior chamber depth and an approximately 4-5 mm continuous curvilinear capsulorhexis was performed.

(4) Removal of the nucleus and the cortex, and treatment with the cytoskeletal drug/vehicle: Hydrodissection with balanced salt solution (BSS) mixed with 1:1,000,000 epinephrine (Anpro Pharmaceutical, Arcadia, Calif.) was performed by inserting a 19-gauge hydrodissection tip into the opening of the anterior capsule through the corneal wound, and endocapsular lens extraction was performed by phacoemulsification and irrigation/aspiration with the BSS mixture. After lens nucleus and major cortical materials had been removed from the capsular bag by the irrigation/aspiration with ultrasound tip, some additional viscoelastic was injected into the anterior chamber. Thereafter, a needle was inserted through the corneal incision and the viscoelastic in the anterior chamber and into the capsular bag, followed by an injection of 1 ml of drug/vehicle. Identification of the drug/vehicle solution was masked to the surgeon. The injected solution (drug/vehicle) was allowed to sit in contact with the capsule for 10 minutes. Thereafter, irrigation/aspiration of the capsular bag was conducted with BSS mixture plus drug/vehicle (same concentration as bolus injection) for 5 min by using the routine irrigation/aspiration tip with a two-way syringe. During the 5-min irrigation/aspiration, considerable care was taken to remove all observable lens cortical material from the capsular bag and to polish the lens capsule.

5. Wound closure: At the completion of the procedures above, the corneal incision was closed with 10-0 nylon sutures as needed.

6. Cornea, anterior chamber, iris and lens capsular bag were observed immediately following surgery to evaluate acute responses of these tissues to the drug/vehicle.

7. Enucleation and lens tissue preparation: Following extracapsular lens extraction and SLE, enucleation was performed. The rabbit was then sacrificed with an overdose of pentobarbital. The enucleated eye was sectioned in the coronal plane just anterior to the equator. After carefully removing the cornea, iris and vitreous, the lens capsular bag was maintained in its original position, and the zonules was not touched. The anterior sclera and attached tissues were further cut from the edge of the coronal section so that a 2-4 mm ciliary body—sclera ring including the lens capsular bag and the zonules remained. The lens capsular bag and the surrounding ciliary body—sclera ring were fixed in 4% paraformaldehyde and stored in a refrigerator. The lens capsule was then observed by light microscopy and recorded by an attached digital camera. Based on images, the interior surface of the capsular bag was divided into two types of areas: (A) the dirty area that had dense residual materials (e.g, residual cortex and associated lens epithelial cells) and (B) the relatively clean area that included the area with scattered cells and the area without cells.

Results

Area A represents the area where the irrigation/aspiration tip did not reach during surgery and area B represents the area where the irrigation/aspiration tip reached during surgery. Area A occupied about 5-20% of the entire capsular surface while Area B occupied about 80-95%.

For Area A, there was no difference between the latrunculin-B-treated eye and the vehicle-treated eye. For Area B, the latrunculin-B-treated eye had fewer residual lens epithelial cells than the vehicle-treated eye. Therefore, 2 μM latrunculin-B facilitated the clearance of residual lens epithelial cells during the extracapsular lens extraction.

### EXAMPLE 2

### In Vivo Rabbit Eye Model and Human Lens Capsular Bag Culture Model

Section one below describes methods that can be used to test the effects of single treatments of an actin-disrupting compound (e.g., latrunculin-A or latrunculin-B) or H-7 before and during lens capsule polishing on clearance of residual lens epithelial cells. Section two below describes methods that can be used to test the effects of chronic topical treatments with an actin-disrupting compound (e.g., latrunculin-A or latrunculin-B) or H-7 on PCO formation or lens epithelial cells' division and migration.

For example, the effects of latrunculin-B and H-7, either used separately or concurrently, can be tested. In section-one protocols, 2 μM latrunculin-B and/or 300 μM H-7 can be used initially as single intra-capsular treatments during surgery in both the live rabbit eye and the donated human eye. The concentration of latrunculin-B or H-7 may be reduced to 0.5 μM or 100 μM, respectively, in the second step (see below). In section-two protocols, the live rabbit eye may receive an intra-capsular treatment with the same concentration of the drug(s) as used in section-one protocols immediately after surgery. Thereafter, the rabbit can be treated topically with 0.01% latrunculin-B and/or 5% H-7 for 4 weeks (see below). In the cultured human lens capsular bag, 0.5 μM latrunculin-B or 100 μM H-7 may be used. The drug concentrations for cultured lens capsules may be adjusted when necessary. All these concentrations of the drugs significantly increased outflow facility in living monkeys (Tian B, et al. Arch Ophthalmol. 116:633-643, 1998; Peterson J A, et al. Exp Eye Res 70:307-313, 2000; Sabanay I, et al. Exp Eye Res. 2005; In press; Tian B, et al. Arch Ophthalmol. 122:1171-1177, 2004; Okka M, et al. Arch Ophthalmol. 122:1482-1488, 2004), indicating that they induce structural changes in the trabecular meshwork. The concentrations required for the drugs to alter the actin cytoskeleton in different cells, including bovine aortic endothelial cells, human trabecular meshwork cells, and chicken lens epithelial cells, are quite similar, (Tian B, et al. Arch Ophthalmol. 116:633-643, 1998; Peterson J A, et al. Invest Ophthalmol Vis Sci. 40:931-941, 1999; Liu X, et al. Mol Vision. 7:145-153, 2001; Volberg T, et al. Cell Motil Cytoskel. 29:321-338, 1994; Cai S, et al. Mol Vision. 6:132-143, 2000). Additionally, anterior chamber exchange and continuous intracameral infusion with the outflow facility-effective concentrations of the drug for 45-90 min did not induce any observable damage to the lens, lens capsule or corneal endothelium and other adjacent tissues as assessed by biomicroscopy and/or light/electron microscopy (Tian B, et al. Arch Ophthalmol. 116:633-643, 1998; Peterson J A, et al. Invest Ophthalmol Vis Sci. 40:931-941, 1999; Peterson J A, et al. Exp Eye Res 70:307-313, 2000; Sabanay I, et al. Exp Eye Res. 2005; In press; Tian B, et al. Curr Eye Res. 22:109-120, 2001; Sabanay I, et al. Exp Eye Res. 2005; In press; Tian B, et al. Arch Ophthalmol. 122:1171-1177, 2004; Okka M, et al. Arch Ophthalmol. 122:1482-1488, 2004). The combination of H-7 and latrunculin-B is expected to better enhance separations of residual cells from each other and from the posterior capsule.

In section one (single endocapsular treatment), 50 albino rabbits may be used. In the first step, 25 experimental animals may be divided into 5 groups (5 for each): Group 1 may receive 300 μM H-7; Group 2 may receive 2 μM latrunculin-B; Group 3 may receive 300 μM H-7 plus 2 μM latrunculin-B; Group 4 may receive drug-free vehicle; and Group 5 may receive vehicle plus 0.25% DMSO. In the second step, there are two options. If the treatment with 2 μM latrunculin-B and/or 300 μM H-7 thoroughly removes residual lens epithelial cells from the capsular bag in the first step, the experiment may be repeated with the other 25 rabbits, during which 0.5 μM latrunculin-B and/or 100 μM H-7 may be used to determine if lower dose works (Option 1). Otherwise, higher concentrations (e.g., 5 μM latrunculin-B and/or 500 μM H-7) may be used (Option 2). An alternative protocol for Option 2 is that low power ultrasound may be added following each treatment as in the first step to determine if the low power ultrasound is helpful to break down the drug-weakened cellular adhesions. Each rabbit may provide two drug(s)-treated eyes or two vehicle (±0.25% DMSO)-treated eyes (so that there are 10 eyes for each group), and may be euthanized after enucleation immediately after surgery.

In section two (chronic topical treatments; twice daily for 4 weeks), 45 albino rabbits may be used. Experimental animals may be divided into 5 groups (9 for each): Group 1 may receive 5% topical H-7; Group 2 may receive 0.01% topical latrunculin-B; Group 3 may receive 5% topical H-7 plus 0.01% latrunculin-B; Group 4 may receive drug-free vehicle; and Group 5 may receive vehicle plus 0.25% DMSO. Each rabbit may provide one drug(s)-treated eye or one vehicle (±0.25% DMSO)-treated eye and the opposite eye may be maintained untouched for better quality of life during the 4-week experimental period.

Donated human eye (after removing comeoscleral disc for transplantation) may be obtained from the Rotary Aravind International Eye Bank, located at Aravind Eye Hospital, Madurai. In section one (single endocapsular treatment during surgery), five experimental groups may be employed for each step as in section one rabbits and 6 to 9 eyes may be included for each group. Therefore, 60 to 90 human donor eyes are necessary (30 to 45 for each step). In section two (long term treatment after surgery), 30 to 45 human donor eyes may be used. Experimental eyes may be divided into 5 groups (6 to 9 for each) as above.

Section One

A. Rabbits:

1. Slit lamp examination (SLE) is performed before each study to exclude rabbits that have ocular inflammation or other abnormalities.

2. Non-steroidal anti-inflammation drug (NSAID) pre-treatment: Intramuscular injection (i.m.) with carprofen (1-5 mg/kg) or flunixin (1.0 mg/kg) is made once daily for one week.

3. Animal anesthesia: A pre-medication of 20-50 mg/kg i.m. ketamine, 2-5 mg/kg i.m. xylazine, and 0.01-0.05 mg/kg buprenorphine SQ is given. Animals are masked with isoflurane to relax for intubation and then maintained at 1-5% isoflurane in O₂.

4. Extracapsular lens extraction:

(1) Eye preparations: Dilate the pupil of the surgical eye with 1% cyclopentolate (Cyclogyl™), 0.3% flurbiprofen (Ocufen [symbol] ), 2.5% phenylephrine, and 1% tropicamide (Mydriacyl™) (4 times at five-minute intervals); trim eyelashes; disinfect the ocular area with povidone iodine; and insert a wire lid speculum to retract the lids.

(2) Clear cornea approach: A corneal incision is made superiorly with a 3.0-3.2 mm keratome.

(3) Anterior capsulotomy: Viscoelastic (Provisc™) is injected to maintain anterior chamber depth and an approximately 4-5 mm continuous curvilinear capsulorhexis is performed.

(4) Removal of the nucleus and the cortex, and treatment with the cytoskeletal drug/vehicle: Hydrodissection with BSS mixed with 1:1,000,000 epinephrine (Anpro Pharmaceutical, Arcadia, Calif.) is performed by inserting a 19-gauge hydrodissection tip into the opening of the anterior capsule through the corneal wound, and endocapsular lens extraction is performed by phacoemulsification and irrigation/aspiration with the BSS mixture. Although it is not clear if heparin interferes with latrunculins or H-7's effect on the actin cytoskeleton, heparin is not included in the BSS mixture because it increases stress fiber formation (Ehrlich H P, et al. Exp Cell Res. 164:154-162, 1986). To reduce the inflammation induced by the breakdown of blood-aqueous barrier during surgery, pre-treatment with NSAID is conducted as described above. When lens nucleus and major cortical materials have been removed from the capsular bag by the irrigation/aspiration with ultrasound tip, some viscoelastic is injected into the anterior chamber. Thereafter, a needle is inserted through the corneal incision and the viscoelastic in the anterior chamber and into the capsular bag, followed by an injection of 1 ml of drug/vehicle. The injected solution (drug/vehicle) is allowed to sit in contact with the capsule for 10 minutes. Thereafter, irrigation/aspiration of the capsular bag is conducted with BSS mixture plus drug/vehicle (same concentration as bolus injection) for 5 min by using the routine irrigation/aspiration tip with a two-way syringe. During the 5-min irrigation/aspiration, considerable care should be taken to remove all observable lens cortical material from the capsular bag and to polish the lens capsule.

5. Wound closure: At the completion of the procedures above, the corneal incision is closed with 10-0 nylon sutures as needed.

6. SLE: Cornea, anterior chamber, iris, and lens capsular bag are observed immediately following surgery to evaluate acute responses of these tissues to the drug/vehicle.

7. Enucleation and lens tissue preparation: Following extracapsular lens extraction and SLE, enucleation is performed. The rabbit is then sacrificed with an overdose of pentobarbital. The enucleated eye is sectioned in the coronal plane just anterior to the equator. After carefully removing the cornea, iris, and vitreous, the lens capsular bag is maintained in its original position and the zonules are not touched. The anterior sclera with attached tissues is further cut from the edge of the coronal section so that a 2-4 mm ciliary body—sclera ring including the lens capsular bag and the zonules remains. The lens capsular bag and the surrounding ciliary body—sclera ring are fixed in 4% paraformaldehyde and stored in a refrigerator. The lens capsule is observed by light microscopy (LM) before and/or after being stained with hematoxylin and eosin (H&E). The cell area remaining (e.g., % of the cell-free area in the entire capsule) is determined by systematically examining specimens through a grid in the microscope's eyepiece or the number of residual lens epithelial cells is counted if it is small. Alternatively, the LM images are evaluated using Image Analysis Software. Capsular lesions, if any, are graded on a 4-point scale (0: no capsular lesions; 1: mild lesions; 2: moderate lesions and 3: severe lesions). Toxicity study with other parts of the enucleated eye may optionally be done by LM, electron microscopy (EM), specular microscopy, pachymetry, biomicroscopy, and/or electroretinography (Tian B, et al. Arch Ophthalmol. 116:633-643, 1998; Peterson J A, et al. Invest Ophthalmol Vis Sci. 40:931-941, 1999; Peterson J A, et al. Exp Eye Res 70:307-313, 2000; Sabanay I, et al. Exp Eye Res. 2005; In press; Tian B, et al. Curr Eye Res. 22:109-120, 2001; Sabanay I, et al. Exp Eye Res. 2005; In press; Tian B, et al. Arch Ophthalmol. 122:1171-1177, 2004; Okka M, et al. Arch Ophthalmol. 122:1482-1488, 2004; Kiland J A, et al. Invest Ophthalmol Vis Sci 43: E-Abstract 3291, 2002; Miller C L, et al. Invest Ophthalmol Vis Sci 45: E-Abstract 444, 2004). Biomicroscopy of the rabbit eye following surgery/treatment(s) may optionally be performed to provide safety evaluation.

Currently, phacoemulsification with ultrasound represents the standard procedure for removing the lens nucleus and the major portion of the cortex during cataract surgery. However, although the ultrasound irrigation/aspiration technique is experimentally more effective in removing lens epithelial cells than irrigation/aspiration alone (Mathey C F, et al. J Cataract Refract Surg. 20:64-69, 1994), it is not used clinically for lens capsule polishing, since high ultrasound power or direct tissue contact by the ultrasound tip may damage the lens capsule and/or other adjacent ocular tissues. Low power ultrasound without directly contacting the capsule during polishing may be safe, but may not be sufficient to thoroughly remove normal residual lens epithelial cells. However, cytoskeletal drug treatment to weaken cellular adhesions followed by low power ultrasound without direct tip-capsule contact (see below), may help the cells to separate from the capsule or neighboring cells without damaging any ocular tissues. Therefore, we anticipate that cytoskeletal drugs and low power ultrasound may be used together to remove residual lens epithelial cells more effectively during cataract surgery. Combining the two modalities may avoid side effects on adjacent tissues by reducing the dose/power required of each one. Nevertheless, the use of ultrasound following drug treatment may not be necessary, because current routine irrigation/aspiration during capsule polishing may have sufficient power to completely remove the drug-weakened residual lens epithelial cells.

If low power ultrasound is used in the protocol for section one rabbits, the alternative procedures are as follows: Ten min after the drug/vehicle treatment, irrigation/aspiration with the BSS mixture plus drug/vehicle is performed for 2 min initially by inserting the ultrasound tip into the center of the capsular bag and holding it still (25 ml/min flow; 0 mmHg vacuum; 10% or slightly higher ultrasound power). Although the ultrasound tip does not directly contact the capsule in this method, the 2-min treatment with ultrasound may help to more effectively remove the drug-weakened residual lens epithelial cells per the principle of ultrasonic cleaner. The main mechanism for the cleaning action in an ultrasonic cleaner is actually the energy released from the collapse of millions of microscopic cavitations occurring in the liquid of the cleaner. The energy from cavitation occurring in the liquid in the lens capsular bag may indirectly detach or loosen the drug-weakened residual lens epithelial cells from the capsule and the neighboring cells. Alternatively, the ultrasound tip plus a cannula covered in a silicone sleeve (a so-called Capsule Polisher) may be safe for directly touching the capsule if an even lower ultrasound energy is used (e.g. 5% power). This technique did not damage the lens capsule and other adjacent tissues when 10% ultrasound power was used (Hepsen I F, et al. J Cataract Refract Surg. 23:1572-1576, 1997). Following the 2-min ultrasound treatment, a 3-min irrigation/aspiration is performed using a two-way syringe with a capsule polisher for the inferior quadrants and a j-or similar cannula with a silicone sleeve for the superior quadrants. During the 3-min irrigation/aspiration, considerable care should be taken to remove all observable lens cortical material from the capsular bag and to polish the lens capsule.

B. Donated Human Eyes:

1. Surgery and treatment: Cadaver eye where the comeoscieral button has been removed for corneal transplantation is used. However, if ultrasound is used in second step as described above, 30 to 45 donated human eyes with intact corneas may be obtained from the eye bank, since a closed or semi-closed anterior chamber is needed for ultrasound to function effectively. Sham cataract surgery is conducted similarly to the procedures as described in the protocol for rabbits above with some modification. Briefly, the globe is fixed in a specific device, and the iris tissue is removed such that the lens zonular complex is well visualized. A 5-6 mm continuous curvilinear capsulorhexis is performed using a 26-gauge needle. Cortical cleaving hydrodissection and hydrodelineation is then performed. An endocapsular ring is implanted in the bag. The purpose of this ring is to stretch the bag and also enable dissection of the bag following cataract removal. One pole of the nucleus is prolapsed out of the capsular bag using a Sinskey hook and the rest wheeled out gently. Manual irrigation/aspiration is performed using the Simcoe cannula to remove lens fibers. The drug/vehicle treatment and capsule polishing is conducted as in the rabbit protocol.

2. Lens tissue preparations: The capsular bag is dissected from its zonular attachments with Vannas scissors and fixed in 4% cold paraformaldehyde and stained using H&E. The lens capsule is observed by LM as described in the rabbit protocol above.

In both the rabbit eye model and the human capsular bag culture model described above, it is expected that treating the lens capsule with an actin-disrupting compound and/or H-7 (e.g., 300 μM H-7, 2 μM latrunculin-B, or 300 μM H-7+2 μM latrunculin-B) will lead to more lens epithelial cells be removed in comparison to treating the lens capsule with a control vehicle (e.g., drug-free vehicle or drug-free vehicle+0.25% DMSO) as determined by LM.

Section Two

A. Rabbits:

1. SLE and NSAID pretreatment is same as that in section one rabbits.

2. Baseline SLE videography and photography images of rabbit eyes are collected before surgery. The SLE videography and photography are performed. The entire lens and the posterior capsule of the lens are imaged.

3. Animal anesthesia is same as above.

4. Extracapsular lens extraction is conducted as in the rabbits in section one, but drug/vehicle treatment is not conducted until the surgery is finished. Briefly, hydrodissection with BSS mixed with 1:1,000,000 epinephrine is performed by inserting a 19-gauge hydrodissection tip into the opening of the anterior capsule through the corneal wound, and endocapsular lens extraction is performed by phacoemulsification and irrigation/aspiration with the BSS mixture. When lens nucleus and major cortical materials have been removed from the capsular bag by the ultrasound irrigation/aspiration tip, 5-min irrigation/aspiration is conducted by using the regular irrigation/aspiration tip with a two-way syringe. During the 5-min irrigation/aspiration, considerable care should be taken to remove all observable lens cortical material from the capsular bag and to polish the posterior capsule. No IOLs are implanted after polishing because inhibitory effect of IOLs on the migration of lens epithelial cells may confound the results. At the completion of the procedures above, the corneal incision is closed with 10-0 nylon sutures as needed. Thereafter, a needle is inserted through the corneal incision and into the capsular bag, followed by an injection of 1 ml of drug/vehicle.

5. Treatment immediately after surgery: Inject gentamycin (20 mg, 0.25-0.5 ml) and methylprednisolone acetate (Depo-medrol; 20 mg; 0.25-0.5 ml) subconjunctivally, and then instill some polymixin/neomycin antibiotic ointment in the conjunctival sac and close the eyelids.

6. Postoperative care: The rabbit is closely observed before recovery from anesthesia for any signs of discomfort, and then returned to the cage. In addition to specific biomicroscopic observations (see below), the operated eye and general physical condition are observed during daily treatment for 4 weeks to make sure the rabbit is recovering from the surgery.

7. Routine postoperative treatment: Routine topical treatments are performed once daily at noon, including administering one drop of 1% tropicamide and 10% phenylephrine for about 2 weeks. Intramuscular carprofen (1-5 mg/kg) or flunix (1.0 mg/kg) is administered once daily for two weeks (1-5 mg/kg i.m.).

8. Cytoskeletal drug/vehicle treatments: Twenty microliters (2×10 μl) of cytoskeletal drug/vehicle solution (the concentration for each drug as above) is applied twice daily for 4 weeks (starting from the second day following surgery) at 7:00-8:00 and 16:00-17:00, with an interval of 60 sec between drops. Alternately, 10 μl of cytoskeletal drug/vehicle solution is administered by a bolus intracameral injection at predetermined intervals (e.g., once every three days).

9. Postoperative SLE, videography and photography: Perform SLE 1-2 days after the operation to make sure that the eye is free of acute complications, and then repeat SLE every week to observe the eye's recovery and to evaluate PCO formation. PCO is graded on a four-point scale: 0: clear, no visible proliferative tissue on the peripheral or central posterior capsule; 1: mild, proliferative tissue in the periphery only; 2: moderate, sparse proliferative tissue on both the peripheral and central capsule; 3: dense, diffuse and thick opacification on entire capsule (Hepsen I F, et al. J Cataract Refract Surg. 23:1572-1576, 1997; Inan ÜÜ, et al. Graefe's Arch Clin Exp Ophthalmol. 239:693-697, 2001). Videography and photography images of the lens capsular bag are collected 2 and 4 weeks after surgery. The images are evaluated with the four-point scale as above. The comparison of the ranks obtained by the four-point scale between two groups (e.g., H-7 vs. vehicle, latrunculin-A vs. vehicle; latrunculin-B vs. vehicle, or latrunculin-B+H-7 vs. vehicle) is analyzed with Mann-Whitney Test. Comparisons of the ranks by the four-point scale among the five groups is analyzed with Kruskal-Wallis Test.

10. LM: After final SLE, videography and photography, the rabbits are sacrificed with an overdose of pentobarbital and their globes are enucleated. The lens tissues and its adjacent tissues for LM are prepared as described in section one.

B. Donated Human Eyes:

1. Extracapsular lens extraction: Same as that in section one for donated human eye, but no endocapsular ring implantation or drug/vehicle treatment is necessary during surgery.

2. Capsular bag culture and treatment: Following surgery, the capsular bag is dissected free from the zonules and secured on a sterile polymethylmethacrylate petri dish. Six to eight entomological pins (Watkins and Doncaster, Kent, UK) are inserted through the edge of the capsular bag to retain its circular shape (Liu C S, et al. Invest Ophthalmol & Vis Sci. 37:906-914, 1996). Alternatively, the donated human eye is sectioned in the coronal plane just anterior to the equator following surgery. After removing the cornea and iris (if present) and vitreous, the lens capsular bag is maintained in its original position, and the zonules is not touched. The anterior sclera and attached tissues are further cut from the edge of the coronal section so that a 2-4 mm ciliary body—sclera ring including the lens capsular bag and the zonules remain. This modified method provides a more physiological way to retain the circular shape of the lens capsular bag during culture. The lens capsular bag located in the center of the ciliary body—sclera ring is cultured in a sterile polymethylmethacrylate petri dish. Cultures are maintained at 37° C. in a 5% CO₂ atmosphere in Eagle's minimum essential medium with cytoskeletal drug/vehicle (the concentration for each drug as described above) supplemented with 10% fetal calf serum and 50 mg/L gentamycin. The maintenance medium with drug/vehicle is replaced every 3 to 4 days. This system can last as long as 100 days (Liu C S, et al. Invest Ophthalmol & Vis Sci. 37:906-914, 1996).

3. PCO evaluation: Observations of proliferation of lens epithelial cells and capsular wrinkling and tensing are performed every day using phase-contrast and dark-field microscopy, and appearance of each capsular bag is recorded during each observation. The iris diaphragm of the microscope is closed down to give a graded illumination, which can show the contours of the wrinkled areas clearly. The increased capsular tensing, the net result of the progressive capsular folding, can clearly be seen as the inwardly bowed region between entomological pins. Photography images are collected whenever needed. Eventually, PCO is graded on a four-point scale: 0: clear, no visible proliferative tissue on the peripheral or central posterior capsule; 1: mild, proliferative tissue in the periphery only; 2: moderate, sparse proliferative tissue on both the peripheral and central capsules; 3: dense, diffuse and thick opacification on entire capsule (Hepsen I F, et al. J Cataract Refract Surg. 23:1572-1576, 1997; Inan ÜÜ, et al. Graefe's Arch Clin Exp Ophthalmol. 239:693-697, 2001). The comparison of the ranks obtained by the four-point scale between two groups (e.g., H-7 vs. vehicle, latrunculin-B vs. vehicle (with 0.25% DMSO), or latrunculin-B+H-7 vs. vehicle (with 0.25% DMSO)) is analyzed with Mann-Whitney Test. Comparisons of the ranks by the four-point scale among all groups is analyzed with Kruskal-Wallis Test.

In both the rabbit eye model and the human capsular bag culture model described above, it is expected that chronic treatment of the lens capsule with an actin-disrupting compound and/or H-7 (e.g., 5% H-7, 0.01% latrunculin-B, or 5% H-7+0.01% latrunculin-B) will prevent the formation or reduce the severity of PCO in comparison to treatment with a control vehicle (e.g., drug-free vehicle or drug-free vehicle+0.25% DMSO).

The present invention is not intended to be limited to the embodiments and examples described above, but rather to encompass all such variations and modifications as come within the scope of the appended claims.

We claim:

1. A method for reducing posterior capsular opacification in a human or non-human animal, the method comprising the steps of:
(a) providing access to lens epithelial cells attached to an interior surface of a lens capsule of an eye in the human or non-human animal; and
(b) contacting said lens epithelial cells with a compound selected from the group consisting of an actin-disrupting compound and H-7 in an amount sufficient to reduce posterior capsular opacification.

2. The method of claim 1, wherein the actin-disrupting compound is selected from the group consisting of a latrunculin, swinholide-A, jasplakinolide, and a cytochalasin.

3. The method of claim 1, wherein the actin-disrupting compound is selected from the group consisting of latrunculin-A and latrunculin-B.

4. The method of claim 1, wherein the method is for reducing posterior capsular opacification in a human.

5. The method of claim 1, wherein the actin-disrupting compound is provided in a solution.

6. The method of claim 5 further comprising the step of washing the capsule to remove lens epithelial cells.

7. The method of claim 1, wherein the actin-disrupting compound is provided on the outside surface of an ocular implant.

8. The method of claim 7, wherein the ocular implant is an artificial intraocular lens.

9. The method of claim 1, wherein the actin-disrupting compound is provided in a sustained release formulation.

10. The method of claim 1, further comprising the steps of:
removing lens epithelial cells, lens nucleus, and lens cortex from the capsule; and
inserting an ocular implant into the lens capsule.

11. The method of claim 10, wherein the ocular implant is an artificial intraocular lens.

12. The method of claim 10, wherein the ocular implant is coated with a compound selected from the group consisting of an actin-disrupting compound and H-7.

13. A kit comprising an ocular implant and a composition that comprises an actin-disrupting compound or H-7.

14. The kit of claim 13, wherein the actin-disrupting compound is selected from the group consisting of a latrunculin, swinholide-A, jasplakinolide, and a cytochalasin.

15. The kit of claim 13, further comprising a viscoelastic material.

16. The kit of claim 13, further comprising a dye.

17. The kit of claim 13, further comprising an instruction manual for using the composition in a manner so as to cause the composition to contact lens epithelial cells attached to an interior surface of a lens capsule of an eye.

18. An ocular implant coated with a compound selected from the group consisting of an acting-disrupting compound and H-7.

19. The ocular implant of claim 18, wherein the ocular implant is an artificial intraocular lens.

20. The ocular implant of claim 18, wherein the implant is coated with an actin-disrupting compound selected from the group consisting of a latrunculin, swinholide-A, jasplakinolide, and a cytochalasin.
